(19) United States
(12) Patent Application Publication
(10) Pub. No.: US 2011/0294815 A1
(43) Pub. Date: Dec. 1, 2011

(54) PROSTACYCLIN ANALOGS

(76) Inventor: Scott L. Harbeson, Cambridge, MA (US)

(21) Appl. No.: 13/001,543

(22) PCT Filed: Jun. 26, 2009
(86) PCT No.: PCT/US09/03801

§ 371 Date: Mar. 31, 2011

Related U.S. Application Data
(62) Division of application No. 61/133,306, filed on Jun. 27, 2008.

(51) Int. Cl. A61K 31/192; A61K 31/506; A61K 31/422; A61K 31/505; A61K 31/496; A61K 31/4985; A61K 31/53; A61P 9/12; A61P 37/06; A61P 9/10; A61P 9/04; A61P 9/00; A61P 1/04; A61P 35/00; A61P 13/12; A61P 19/04; A61P 3/10; A61P 7/02; C07C 59/72

(52) U.S. Cl. 514/243; 562/466; 514/569; 514/269; 514/380; 514/274; 514/252.16; 514/250

(57) ABSTRACT

This invention relates to novel prostacyclin analogs and pharmaceutically acceptable salts thereof. More specifically, the invention relates to novel prostacyclin analogs that are derivatives of treprostinil. This invention also provides compositions comprising a compound of this invention and a carrier and the use disclosed compounds and compositions in methods of treating diseases and conditions that are beneficially treated by administering analogues of prostacyclin, such as treprostinil.

### RELATED APPLICATION

This application claims the benefit of U.S. Provisional Application No. 61/133,306, filed on Jun. 27, 2008. The entire teachings of the above application(s) are incorporated herein by reference.

### BACKGROUND OF THE INVENTION

Treprostinil, also known as 9-deoxy-3,7-(1′,3′-interphenylene)-2′,9-methano-3-oxa-4,5,6-trinor-13,14-dihydroprostaglandin F1 sodium salt, and as (1R,2R,3aS,9aS)-[[2,3,3a,4,9,9a-Hexahydro-2-hydroxy-1-[(3S)-3-hydroxyoctyl]-1H-benz[f]inden-5-yl]oxy]acetic acid monosodium salt, acts directly on pulmonary and systemic arterial vascular beds causing vasodilation and inhibition of platelet aggregation.

Treprostinil is currently approved for the treatment of pulmonary arterial hypertension (PAH) in patients with NYHA Class II-IV symptoms to diminish symptoms associated with exercise. Treprostinil is also in phase. II clinical trials for treatment of PAH associated with idiopathic pulmonary fibrosis (http://clinicaltrials.gov/ct2/show/NCT00703339) and for critical limb ischemia (http://clinicaltrials.gov/ct/show/NCT00445159).

Common adverse events associated with treprostinil include infusion site pain and reaction, diarrhea, jaw pain, edema, vasodilatation, headache and nausea. Since clearance in patients with hepatic insufficiency was reduced by up to 80% compared to healthy adults, caution is advised in treating patients with hepatic or renal impairment. (http://www.fda.gov/cder/foi/label/2006/021272S0051b1.pdf). It would be desirable to provide a compound that has the beneficial activities of treprostinil and other benefits, e.g., reduced adverse side effects, with a decreased metabolic liability, to further extend its pharmacological effective life, enhance patient compliance and, potentially, to decrease population pharmacokinetic variability and/or decrease its potential for dangerous drug-drug interactions.

### SUMMARY OF THE INVENTION

This invention relates to novel prostacyclin analogs and pharmaceutically acceptable salts thereof.

More specifically, the invention relates to novel prostacyclin analogs that are derivatives of treprostinil. This invention also provides compositions comprising a compound of this invention and a carrier and the use disclosed compounds and compositions in methods of treating diseases and conditions that are beneficially treated by administering analogues of prostacyclin, such as treprostinil.

### DETAILED DESCRIPTION OF THE INVENTION

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of treprostinil will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

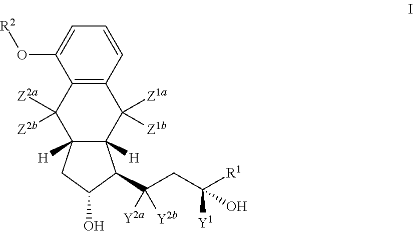

or a pharmaceutically acceptable salt thereof, wherein:

each Z variable is independently selected from hydrogen and deuterium;

each Y variable is independently selected from hydrogen and deuterium;

R¹ is a pentyl group having zero to eleven deuterium substituents; and

R² is selected from —CH₂CO₂H, —CH(D)CO₂H, and —CD₂CO₂H; provided that when each Z variable is hydrogen, each Y variable is hydrogen and R² is CH₂CO₂H, then R′ has at least one deuterium substituent.

The following are various embodiments of the Formula I compounds.
(a) R¹ is a pentyl group having three deuterium at the terminal methyl position and zero to eight additional deuterium substituents.
(b) Each R¹ methylene carbon independently has zero or two deuterium.
(c) R′ is selected from (CH₂)₄CD₃, (CH₂)₃CD₂CD₃, (CH₂)₂(CD₂)₂CD₃, and CD₂(CH₂)₃CD₃.
(d) R′ is selected from (CH₂)₃CD₂CD₃, (CH₂)₂(CD₂)₂CD₃, and CD₂(CH₂)₃CD₃; and R² is selected from CH₂CO₂H and CD₂CO₂H.
(e) Y¹ is deuterium.
(f) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; and Y¹ is deuterium.
(g) Each Y² variable is deuterium.
(h) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; and each Y² variable is deuterium.
(i) Each Y variable is deuterium.
(j) Z^{1a} and Z^{1b} are each deuterium or Z^{2a} and Z^{2b} are each deuterium.
(k) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; Z^{1a} and Z^{1b} are each deuterium or Z^{2a} and Z^{2b} are each deuterium.
(l) Z^{1a} and Z^{1b} are each deuterium or Z^{2a} and Z^{2b} are each hydrogen.
(m) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; Z^{1a} and Z^{1b} are each deuterium or Z^{2a} and Z^{2b} are each hydrogen.
(n) Z^{1a} and Z^{1b} are each hydrogen or Z^{2a} and Z^{2b} are each deuterium.
(o) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; Z^{1a} and Z^{1b} are each hydrogen or Z^{2a} and Z^{2b} are each deuterium.
(p) Each Z variable is deuterium.
(q) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; and each Z is deuterium.
(r) Each Y variable is deuterium and each Z variable is deuterium.
(s) R¹ is selected from (CH₂)₃CD₂CD₃, (CH₂)₂(CD₂)₂CD₃, and CD₂(CH₂)₃CD₃; R² is selected from CH₂CO₂H and CD₂CO₂H; and each Y variable is deuterium and each Z variable is deuterium.
(t) R² is CD₂CO₂H.
(u) R¹ is selected from (CH₂)₃CD₂CD₃, (CH₂)₂(CD₂)₂CD₃, and CD₂(CH₂)₃CD₃; and R² is CD₂CO₂H.
(v) R² is CD₂CO₂H and each Y variable is deuterium.
(w) R′ is selected from (CH₂)₃CD₂CD₃, (CH₂)₂(CD₂)₂CD₃, and CD₂(CH₂)₃CD₃;
(v) R² is CD₂CO₂H and each Y variable is deuterium.
(x) R² is CD₂CO₂H and each Z variable is deuterium.
(y) R¹ is selected from (CH₂)₃CD₂CD₃, (CH₂)₂(CD₂)₂CD₃, and CD₂(CH₂)₃CD₃; and R² is CD₂CO₂H and each Z variable is deuterium.
(z) R² is CD₂CO₂H, each Y variable is deuterium, and each Z variable is deuterium.
(aa) R¹ is selected from (CH₂)₃CD₂CD₃, (CH₂)₂(CD₂)₂CD₃, and CD₂(CH₂)₃CD₃; R² is CD₂CO₂H, each Y variable is deuterium, and each Z variable is deuterium.

In another embodiment of the Formula I compounds, a combination of two or more (e.g., 2, 3, 4 or 5) of the embodiments set forth in (a)-(aa) are present. Example combinations include, but are not limited to: (a) and (b); (c) and (b); (c), (b) and (a); (d) and (c); (d), (c) and (b); (d), (c), (b) and (a); (e) and (d); (e), (d), and (c); (e), (d), (c) and (b); (e), (d), (c), (b) and (a); (g) and (d); (g), (d) and (c); (g), (d), (c) and (b); (g), (d), (c), (b) and (a); (i) and (d); (i), (d) and (c); (i), (d), (c) and (b); (i), (d), (c), (b) and (a); (j) and (d); (j), (d) and (c); (j), (d), (c) and (b); (j), (d), (c), (b) and (a); (l) and (d); (l), (d) and (c); (I), (d), (c) and (b); (l), (d), (c), (b) and (a); (n) and (d); (n), (d) and (c); (n), (d), (c) and (b); (n), (d), (c), (b) and (a); (p) and (d); (p), (d) and (c); (p), (d), (c) and (b); (p), (d), (c), (b) and (a); (r) and (d); (r), (d) and (c); (r), (d), (c) and (b); (r), (d), (c), (b) and (a); (t) and (d); (t), (d) and (c); (t), (d), (c) and (b); (t), (d), (c), (b) and (a); (v) and (d); (v), (d) and (c); (v), (d), (c) and (b); (v), (d), (c), (b) and (a); (x) and (d); (x), (d) and (c); (x), (d), (c) and (b); (x), (d), (c), (b) and (a); (z) and (d); (z), (d) and (c); (z), (d), (c) and (b); and (z), (d), (c), (b) and (a).

In another set of embodiments, any atom in any of the embodiments set forth above is present at its natural isotopic abundance.

Examples of specific compounds of Formula I are shown in Table 1 below.

TABLE 1
Examples of Formula I Compounds

| No. | Y¹ | Y^{2a} | Y^{2b} | Z^{1a} | Z^{1b} | Z^{2a} | Z^{2b} | R¹ | R² |
|---|---|---|---|---|---|---|---|---|---|
| 100 | D | D | D | H | H | H | H | (CH₂)₄CD₃ | CH₂CO₂H |
| 101 | H | H | H | D | D | H | H | (CH₂)₄CD₃ | CH₂CO₂H |
| 102 | H | H | H | H | H | D | D | (CH₂)₄CD₃ | CH₂CO₂H |
| 103 | H | H | H | H | H | H | H | (CH₂)₄CD₃ | CD₂CO₂H |
| 104 | D | D | D | H | H | H | H | (CH₂)₃CD₂CD₃ | CH₂CO₂H |
| 105 | H | H | H | D | D | H | H | (CH₂)₃CD₂CD₃ | CH₂CO₂H |
| 106 | H | H | H | H | H | D | D | (CH₂)₃CD₂CD₃ | CH₂CO₂H |
| 107 | H | H | H | H | H | H | H | (CH₂)₃CD₂CD₃ | CD₂CO₂H |
| 108 | D | D | D | H | H | H | H | (CH₂)₂(CD₂)₂CD₃ | CH₂CO₂H |
| 109 | H | H | H | D | D | H | H | (CH₂)₂(CD₂)₂CD₃ | CH₂CO₂H |
| 110 | H | H | H | H | H | D | D | (CH₂)₂(CD₂)₂CD₃ | CH₂CO₂H |
| 111 | H | H | H | H | H | H | H | (CH₂)₂(CD₂)₂CD₃ | CD₂CO₂H |
| 112 | D | D | D | H | H | H | H | CD₂(CH₂)₃CD₃ | CH₂CO₂H |
| 113 | H | H | H | D | D | H | H | CD₂(CH₂)₃CD₃ | CH₂CO₂H |
| 114 | H | H | H | H | H | D | D | CD₂(CH₂)₃CD₃ | CH₂CO₂H |
| 115 | H | H | H | H | H | H | H | CD₂(CH₂)₃CD₃ | CD₂CO₂H |
| 116 | D | H | H | H | H | H | H | (CH₂)₄CD₃ | CH₂CO₂H |
| 117 | D | H | H | H | H | H | H | (CH₂)₃CD₂CD₃ | CH₂CO₂H |
| 118 | D | H | H | H | H | H | H | (CH₂)₂(CD₂)₂CD₃ | CH₂CO₂H |
| 119 | D | H | H | H | H | H | H | CD₂(CH₂)₃CD₃ | CH₂CO₂H |
| 120 | H | D | D | H | H | H | H | (CH₂)₄CD₃ | CH₂CO₂H |
| 121 | H | D | D | H | H | H | H | (CH₂)₃CD₂CD₃ | CH₂CO₂H |
| 122 | H | D | D | H | H | H | H | (CH₂)₂(CD₂)₂CD₃ | CH₂CO₂H |
| 123 | H | D | D | H | H | H | H | CD₂(CH₂)₃CD₃ | CH₂CO₂H |

or a pharmaceutically acceptable salt of any of the foregoing.

The synthesis of compounds of Formula I can be readily achieved by synthetic chemists of ordinary skill. Relevant procedures and intermediates are disclosed, for instance in Moriarty, R M et al., J Org Chem, 2004, 69:1890-1902; PCT publications WO/2003070163, WO/2000057701; and WO 199921830; U.S. Pat. Nos. 6,441,245; 6,242,482; 4,349,689; and 4,306,075; and European Patent 0 159 784.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

A convenient method for synthesizing compounds of Formula I is depicted in Schemes 1a-1c.

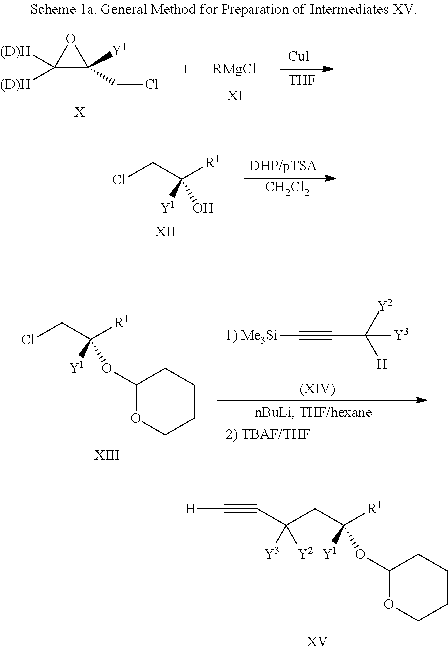

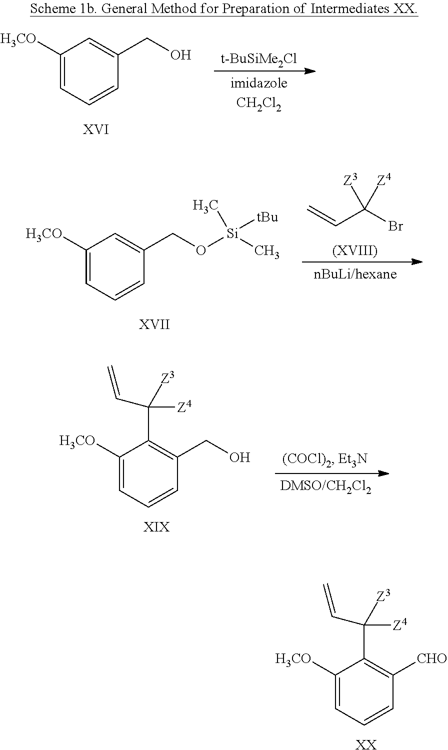

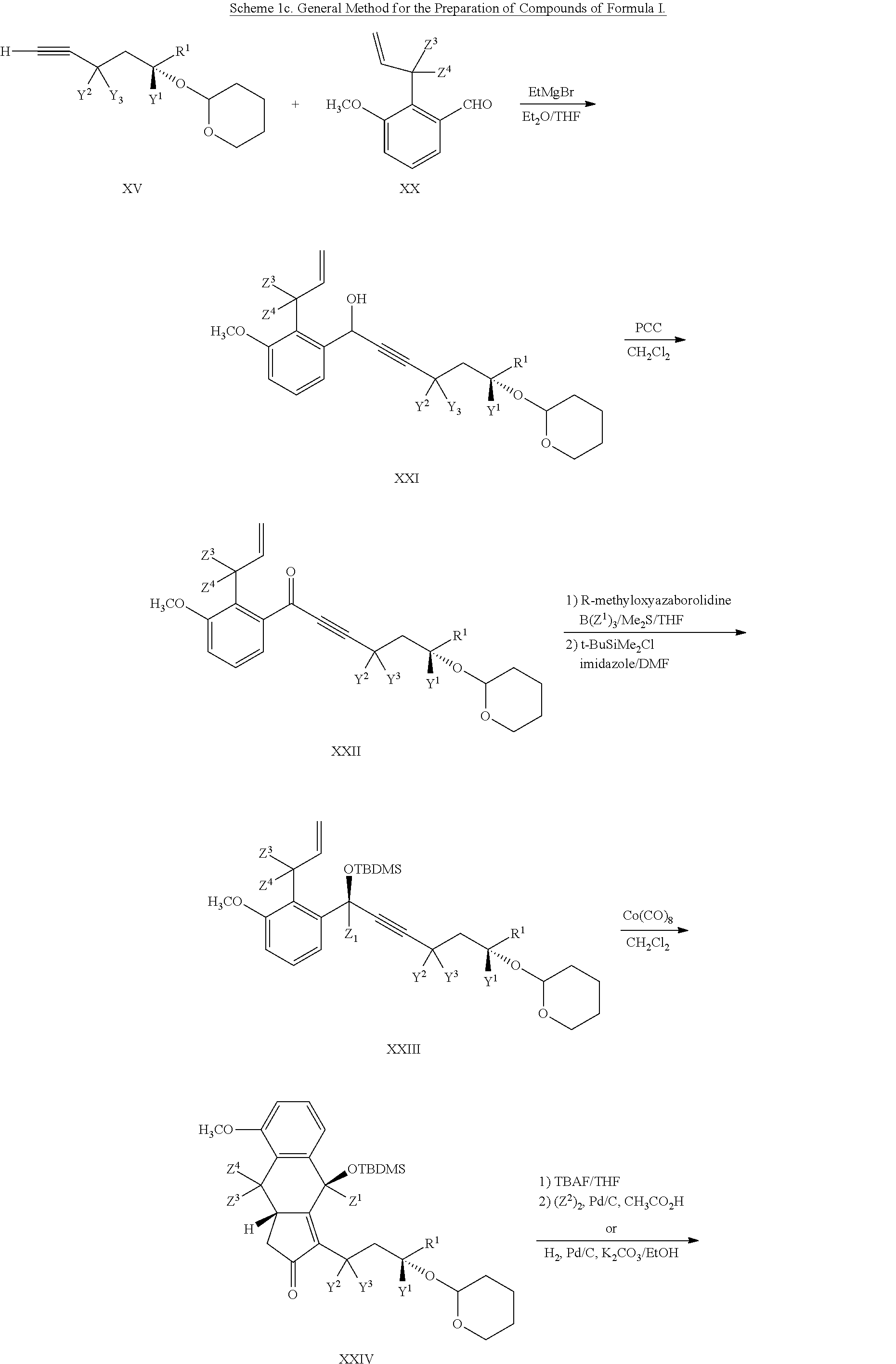

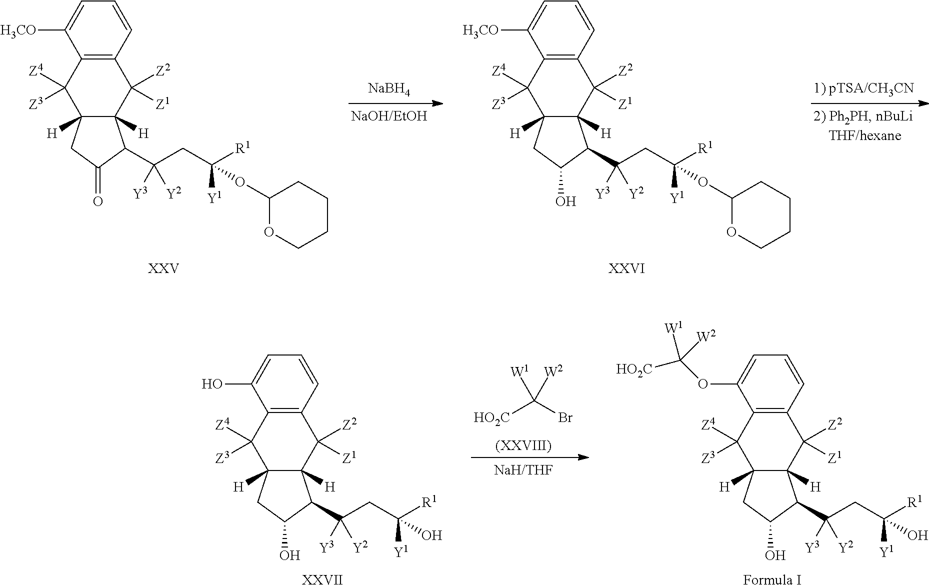

As outlined in Scheme 1a, compound XIII is prepared by treating deuterated or non-deuterated epichlorohydrin X (e.g., Y¹═H) with the appropriately deuterated Grignard reagent XI (wherein R is n-butyl substituted with 0 to 9 deuteriums) in the presence of catalytic CuI followed by protection of the hydroxyl group as a tetrahydropyran ether. Deprotonation of deuterated or non-deuterated silyl alkyne XIV with n-BuLi followed by treatment with XIII and subsequent deprotection of the trimethyl silyl group with tetrabutylammonium fluoride (TBAF) provides XV.

According to the general procedure outlined in Scheme 1b, XIX is prepared by deprotonation of XVII with BuLi followed by treatment with deuterated or non-deuterated allyl bromide XVIII. XVII is readily accessed by treatment of 3-methoxybenzyl alcohol with tert-butyldimethylsilyl chloride and imidazole in dichloromethane. Swern oxidation of XIX provides aldehyde XX.

Compounds of Formula I may be prepared as generally outlined in Scheme 1c. XV on treatment with ethyl magnesium bromide generates the corresponding Grignard reagent, which upon further treatment with aldehyde XX provides XXI. Oxidation of the benzylic alcohol in XXI with pyridinium chlorochromate (PCC) in dichloromethane provides XXII. Asymmetric reduction of the ketone moiety in XXII with R-methyloxazaborolidine and borane or deuterated borane dimethyl sulfide complex, followed by protection of the resulting hydroxyl group with tert-butyldimethylsilyl chloride and imidazole in DMF, provides XXIII. Treatment of XXIII with dicobalt octacarbonyl in dichloromethane provides tricycle XXIV. XXV is prepared by deprotection of the silyl protecting group of XXIV with TBAF followed by hydrogenolysis with deuterium gas in the presence of Pd/C and acetic acid.

Alternatively in cases where Z¹=Hydrogen, XXIV is converted to XXV in one step by treatment with hydrogen gas in the presence of Pd/C and potassium carbonate in ethanol at 90 psi. Reduction of XXV with sodium borohydride in sodium hydroxide and ethanol provides the alcohol XXVI. Deprotection of the tetrahydropyran protecting group in XXVI with p-toluenesulfonic acid in acetonitrile followed by cleavage of methoxy group with lithium diphenylphosphine (prepared in situ from diphenylphosphine and n-BuLi) provides XXVII. The phenolic hydroxyl group in XXVII is alkylated using bromoacetic acid or deuterated bromoacetic and sodium hydride to provide compounds represented by Formula I.

Various deuterated epichlorohydrins may be used in place of compound X in Scheme 1 to provide other compounds of the present invention. Such deuterated epichlorohydrins may be synthesized according to Schemes 2a, 2b and 2c shown below.

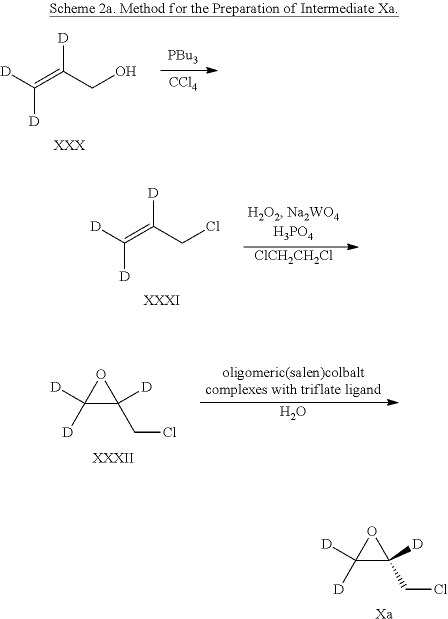

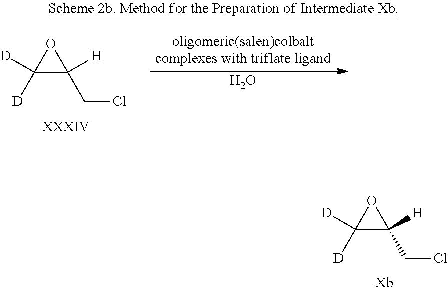

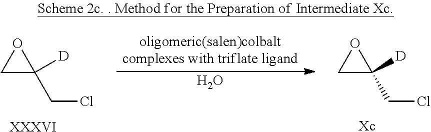

Referring to Scheme 2a, deuterated allyl alcohol XXX (Omichinski, J G et al., Journal of Labelled Compounds and Radiopharmaceuticals, 1988, 25(3):263-275) is converted to XXXI by treatment with carbon tetrachloride and tributylphosphine using a protocol similar to that described in Masato, M et al., J Org Chem, 1985, 50(6):764-768. Epoxidation of XXXI with hydrogen peroxide in the presence of Na tungstate and phosphorous acid under phase transfer conditions and microwave irradiation provides XXXII (Bogdal, D et al., Synthetic Communications, 2005, 35(23):2973-2983). Solvent-free hydrolytic kinetic resolution of XXXII with 0.6 eq water in the presence of oligomeric (salen) cobalt complexes with a triflate ligand provides one deuterated chiral epichlorohydrin Xa (White, D E et al., Tetrahedron Asymmetry, 2003, 14(22):3633-3638).

Referring to Scheme 2b, the preparation of differently deuterated chiral epichlorohydrin Xb is shown using a similar kinetic resolution of XXXIV. Synthesis of XXXIV is described in Kawakami, K., J Org Chem, 1982, 47(18):3581-5.

In Scheme 2c, the preparation of yet another deuterated chiral epichlorohydrin Xe is shown using a similar kinetic resolution of commercially available XXXVI.

Various deuterated Grignard reagents may be used in place of compound XI in Scheme 1. Such deuterated Grignard reagents may be synthesized according to Schemes 3a, 3b and 3c shown below.

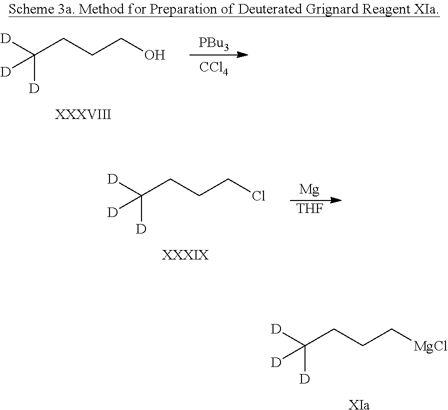

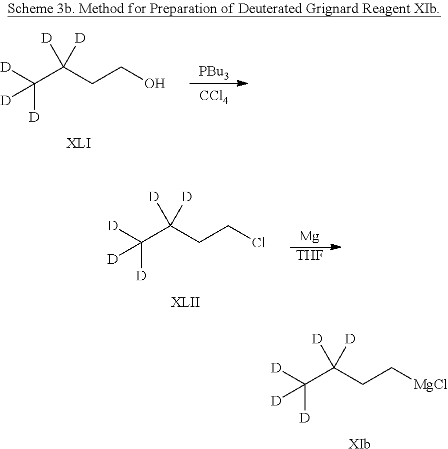

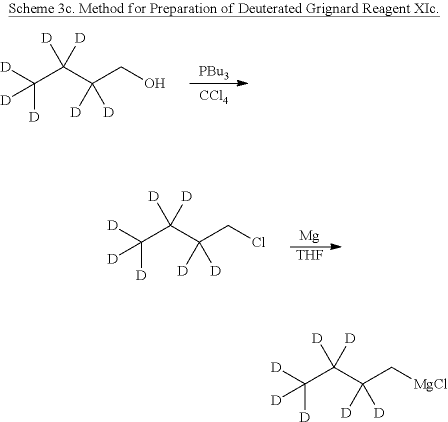

Referring to Schemes 3a-3c, Grignard reagents XIa, XIb and XIc may be prepared from their corresponding chlorides XXXIX, XLII and XLV by treatment with magnesium in THF. The chlorides XXXIX, XLII and XLV may prepared in turn from the commercially available alcohols XXXVIII, XLI and XLIV respectively, by treating with carbon tetrachloride and tributylphosphine using a protocol similar to that described in Masato, M et al., J Org Chem, 1985, 50(6):764-768.

The synthesis of a deuterated trimethylsilyl propyne that may be used in place of compound XLVII in Scheme 1 is depicted in Scheme 4 below.

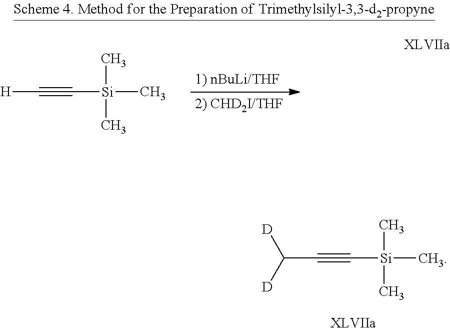

Referring to Scheme 4, compound XLVIIa is prepared by deprotonation of trimethylsilyl acetylene followed by treatment with deuterated methyl iodide using a protocol similar to that described in WO20060069016.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula I (e.g., including any of the formulae herein), or a pharmaceutically acceptable salt of said compound; and an acceptable carrier. In one embodiment, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween; 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the patient therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as treprostinil. Such agents include those indicated as being useful in combination with treprostinil, including but not limited to, those described in WO 2005030187.

Preferably, the second therapeutic agent is an agent useful in the treatment or prevention of a disease or condition selected from pulmonary arterial hypertension and/or ischemia.

In one embodiment, the second therapeutic agent is selected from an endothelin receptor antagonist (ERA) such as bosentan, sitaxsentan sodium or ambrisentan, and/or a phosphodiesterase-5 (PDE-5) inhibitor such as sildenafil citrate, tadalafil or vardenafil hydrochloride hydrate (http://clinicaltrials.gov/ct/show/NCT00325442).

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, to reduce the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., Cancer Chemother. Rep, 1966, 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.2 to 2000 mg per treatment. In more specific embodiments the range is from about 2 to 1000 mg or from 4 to 400 mg or most specifically from 20 to 200 mg per treatment. Treatment typically is administered at a rate of between 0.625 to 1.25 ng/kg/min. The infusion rate can be increased in increments of no more than 1.25 ng/kg/min per week for the first four weeks and then no more than 2.5 ng/kg/min per week for the remaining duration of infusion.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for treprostinil.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of mimicking prostacyclin activity in a cell, comprising contacting a cell with one or more compounds of Formula I or a pharmaceutically acceptable salt of said compounds.

According to another embodiment, the invention provides a method of treating a disease that is beneficially treated by treprostinil comprising the step of administering to a patient in need thereof an effective amount of a compound or a pharmaceutically acceptable salt of said compound or a composition of this invention. Such diseases are well known in the art and are disclosed in, but not limited to the following patents and published applications: U.S. Pat. No. 5,153,222, U.S. Pat. No. 5,028,628, U.S. Pat. No. 5,321,043, U.S. Pat. No. 5,234,953, WO 1999025357, U.S. Pat. No. 4,683,330, WO 2003070163, WO 2005058329, WO 2005058303, and WO 2005099680. Such diseases include, but are not limited to, pulmonary hypertension, transplant rejection, atherosclerosis, congestive heart failure, peripheral vascular disease, critical limb ischemia (CLI), peptic ulcer, cancer, renal failure, connective tissue disease, and diabetic neuropathy.

In one particular embodiment, the method of this invention is used to treat a disease or condition selected from pulmonary arterial hypertension and/or ischemia.

In another particular embodiment, the method of this invention is used to treat a disease or condition selected from pulmonary arterial hypertension.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to said patient one or more second therapeutic agents. The choice of second therapeutic agent may be made from any second therapeutic agent known to be useful for co-administration with treprostinil. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula I or a pharmaceutically acceptable salt of said compound and a second therapeutic agent for treatment of the following conditions: pulmonary hypertension (bosentan, sitaxsentan sodium, ambrisentan, sildenafil citrate, tadalafil or vardenafil hydrochloride hydrate).

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I, or a pharmaceutically acceptable salt of said compound, alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Pharmaceutical Kits

The present invention also provides kits for use to treat pulmonary arterial hypertension and/or ischemia. These kits comprise (a) a pharmaceutical composition comprising a compound of Formula I or a salt thereof, wherein said pharmaceutical composition is in a container; and (b) instructions describing a method of using the pharmaceutical composition to treat pulmonary arterial hypertension and/or ischemia.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage forth. For example, tablet may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In an embodiment of the kits of this invention, the composition comprising the second active agent may be in a vessel or container that is separate from the vessel containing the composition comprising a compound of formula I.

### Example

### Evaluation of Metabolic Stability

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for Compound I and the positive control, 7-ethoxycoumarin (1 μM). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining(ln) vs incubation time]

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

What is claimed is:

1. A compound of formula I: 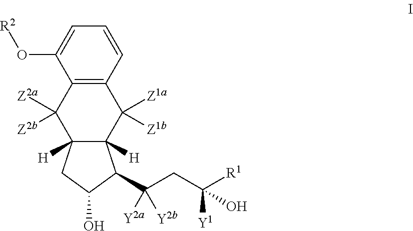
or a pharmaceutically acceptable salt thereof, wherein:
each Z variable is independently selected from hydrogen and deuterium;
each Y variable is independently selected from hydrogen and deuterium;
R¹ is a pentyl group having zero to eleven deuterium substituents; and
R² is selected from —CH₂CO₂H, —CH(D)CO₂H, and —CD₂CO₂H;
provided that when each Z variable is hydrogen, each Y variable is hydrogen and R² is CH₂CO₂H, then R′ has at least one deuterium substituent.

2. The compound of claim 1 where R¹ is a pentyl group having three deuterium at the terminal methyl position and zero to eight additional deuterium substituents.

3. The compound of claim 2 where each R¹ methylene carbon independently has zero or two deuterium.

4. The compound of claim 3 where R¹ is selected from (CH₂)₄CD₃, (CH₂)₃CD₂CD₃, (CH₂)₂(CD₂)₂CD₃, and CD₂(CH₂)₃CD₃.

5. The compound of claim 4 where R¹ is selected from (CH₂)₃CD₂CD₃, (CH₂)₂(CD₂)₂CD₃, and CD₂(CH₂)₃CD₃; and R² is selected from CH₂CO₂H and CD₂CO₂H.

6. The compound of claim 5 where Y¹ is deuterium.

7. The compound of claim 5 where each Y² variable is deuterium.

8. The compound of claim 5 where each Y variable is deuterium.

9. The compound of claim 5 where Z^{1a} and Z^{1b} are each deuterium or Z^{2a} and Z^{2b} are each deuterium.

10. The compound of claim 5 where Z^{1a} and Z^{1b} are each deuterium or Z^{2a} and Z^{2b} are each hydrogen.

11. The compound of claim 5 where Z^{1a} and Z^{1b} are each hydrogen or Z^{2a} and Z^{2b} are each deuterium.

12. The compound of claim 5 where each Z variable is deuterium.

13. The compound of claim 5 where each Y variable is deuterium and each Z variable is deuterium.

14. The compound of claim 5 where R² is CD₂CO₂H.

15. The compound of claim 5 where R² is CD₂CO₂H and each Y variable is deuterium.

16. The compound of claim 5 where R² is CD₂CO₂H and each Z variable is deuterium.

17. The compound of claim 5 where R² is CD₂CO₂H, each Y variable is deuterium, and each Z variable is deuterium.

18. A compound of Formula I selected from the following:

| No. | Y¹ | Y^{2a} | Y^{2b} | Z^{1a} | Z^{1b} | Z^{2a} | Z^{2b} | R¹ | R² |
|---|---|---|---|---|---|---|---|---|---|
| 100 | D | D | D | H | H | H | H | (CH₂)₄CD₃ | CH₂CO₂H |
| 101 | H | H | H | D | D | H | H | (CH₂)₄CD₃ | CH₂CO₂H |
| 102 | H | H | H | H | H | D | D | (CH₂)₄CD₃ | CH₂CO₂H |
| 103 | H | H | H | H | H | H | H | (CH₂)₄CD₃ | CD₂CO₂H |
| 104 | D | D | D | H | H | H | H | (CH₂)₃CD₂CD₃ | CH₂CO₂H |
| 105 | H | H | H | D | D | H | H | (CH₂)₃CD₂CD₃ | CH₂CO₂H |
| 106 | H | H | H | H | H | D | D | (CH₂)₃CD₂CD₃ | CH₂CO₂H |
| 107 | H | H | H | H | H | H | H | (CH₂)₃CD₂CD₃ | CD₂CO₂H |
| 108 | D | D | D | H | H | H | H | (CH₂)₂(CD₂)₂CD₃ | CH₂CO₂H |
| 109 | H | H | H | D | D | H | H | (CH₂)₂(CD₂)₂CD₃ | CH₂CO₂H |
| 110 | H | H | H | H | H | D | D | (CH₂)₂(CD₂)₂CD₃ | CH₂CO₂H |
| 111 | H | H | H | H | H | H | H | (CH₂)₂(CD₂)₂CD₃ | CD₂CO₂H |
| 112 | D | D | D | H | H | H | H | CD₂(CH₂)₃CD₃ | CH₂CO₂H |
| 113 | H | H | H | D | D | H | H | CD₂(CH₂)₃CD₃ | CH₂CO₂H |
| 114 | H | H | H | H | H | D | D | CD₂(CH₂)₃CD₃ | CII₂CO₂H |
| 115 | H | H | H | H | H | H | H | CD₂(CH₂)₃CD₃ | CD₂CO₂H |
| 116 | D | H | H | H | H | H | H | (CH₂)₄CD₃ | CH₂CO₂H |
| 117 | D | II | H | H | H | H | H | (CH₂)₃CD₂CD₃ | CH₂CO₂H |
| 118 | D | H | H | H | H | H | H | (CH₂)₂(CD₂)₂CD₃ | CH₂CO₂H |
| 119 | D | H | H | H | H | H | H | CD₂(CH₂)₃CD₃ | CH₂CO₂H |
| 120 | H | D | D | H | H | H | H | (CH₂)₄CD₃ | CH₂CO₂H |
| 121 | H | D | D | H | H | H | H | (CH₂)₃CD₂CD₃ | CH₂CO₂H |
| 122 | H | D | D | H | H | H | H | (CH₂)₂(CD₂)₂CD₃ | CH₂CO₂H |
| 123 | H | D | D | H | H | H | H | CD₂(CH₂)₃CD₃ | CH₂CO₂H |

or a pharmaceutically acceptable salt of any of the foregoing.

19. A pyrogen-free composition comprising a compound of claim 1 or a pharmaceutically acceptable salt of said compound; and an acceptable carrier wherein the composition is formulated for pharmaceutical use; and the carrier is a pharmaceutically acceptable carrier.

20. (canceled)

21. The composition of claim 19, additionally comprising a second therapeutic agent.

22. The composition of claim 21, wherein the second therapeutic agent is selected from an endothelin receptor antagonist (ERA) and a phosphodiesterase-5 (PDE-5) inhibitor.

23. The composition of claim 22, wherein the second therapeutic agent is selected from bosentan, sitaxsentan sodium, ambrisentan, sildenafil citrate, tadalafil and vardenafil hydrochloride hydrate.

24. (canceled)

25. A method of treating a disease selected from pulmonary hypertension, transplant rejection, atherosclerosis, congestive heart failure, peripheral vascular disease, critical limb ischemia (CLI), peptic ulcer, cancer, renal failure, connective tissue disease, and diabetic neuropathy in a patient in need thereof comprising the step of administering to the patient an effective amount of a composition of claim 19.

26. The method of claim 25, wherein the disease or condition is selected from pulmonary arterial hypertension and ischemia.

27. The method of claim 26, wherein the disease is pulmonary arterial hypertension.

28. A method of causing vasodilation of pulmonary or systemic arterial vascular beds in a patient in need thereof comprising administering to the patient an effective amount of a composition of claim 19.

29. A method of inhibiting platelet aggregation in a patient in need thereof comprising administering to the patient an effective amount of a composition of claim 19.

30. (canceled)

31. (canceled)
